(19) United States
(12) Patent Application Publication
(10) Pub. No.: US 2026/0301556 A1
(43) Pub. Date: Oct. 1, 2026

(54) METHOD, DEVICE AND SYSTEM OF A SENSOR INTEGRATED COMPUTING PLATFORM OF A FIREFIGHTER AIR REPLENISHMENT SYSTEM FOR REMOTE MONITORING AND ACCESS THEREOF

(71) Applicant: Rescue Air Systems, Inc., San Carlos, CA (US)

(72) Inventor: Anthony J. Turiello, Westlake, TX (US)

(73) Assignee: Rescue Air Systems, Inc., San Carlos, CA (US)

(21) Appl. No.: 19/695,448

(22) Filed: Jun. 2, 2026

Related U.S. Application Data
(63) Continuation of application No. 18/197,114, filed on May 15, 2023, now Pat. No. 12,664,870.
(60) Provisional application No. 63/356,996, filed on Jun. 29, 2022.
(60) Provisional application No. 63/359,882, filed on Jul. 11, 2022.

(51) Int. Cl. G08B 21/18; G01N 33/00; G08B 21/02

(52) U.S. Cl. CPC G08B 21/182; G01N 33/0031; G08B 21/02

(57) ABSTRACT

Disclosed are a method, a device and a system of a sensor integrated safety system based computing platform. The computing platform is executed on a data processing device and integrated with a set of sensors associated with components of a safety system In accordance therewith, the data processing device collects a number of parameters of the components of the safety system and/or of access thereof through detection of the number of parameters via the set of sensors. The number of parameters includes one or more parameter(s) related to breathable air from a source within the safety system supplied thereacross via a fixed piping system implemented therein and/or access of the breathable air. The data processing device also monitors the safety system and/or one or more components thereof based on the collected number of parameters.

### CLAIM OF PRIORITY

The present application is a continuation of U.S. patent application Ser. No. 18/197,114, filed May 15, 2023, which claims the benefit of and priority to U.S. Provisional Patent Application No. 63/356,996, filed Jun. 29, 2022, and U.S. Provisional Patent Application No. 63/359,882, filed Jul. 11, 2022, the disclosures of which are incorporated herein by reference in their entireties.

### FIELD OF TECHNOLOGY

This disclosure relates generally to emergency systems and, more particularly, to a method, a device and/or a system of a sensor integrated computing platform of a safety system of a structure for remote monitoring and access thereof.

### BACKGROUND

A structure (e.g., a vertical building, a horizontal building, a tunnel, marine craft) may have a Firefighter Air Replenishment System (FARS) implemented therein. The FARS may have an emergency air fill station therein to enable firefighters and/or emergency personnel access breathable air therethrough. The FARS may have other components relevant to critical functioning thereof. However, tracking parameters (e.g., pressure of the breathable air supplied) of the FARS critical to the functioning and/or the maintenance thereof may be difficult due to a monolithic and/or a standalone implementation of the components of the FARS.

### SUMMARY

Disclosed are a method, a device and/or a system of a sensor integrated computing platform of a safety system of a structure for remote monitoring and access thereof.

In one aspect, a method of a safety system of a structure having a fixed piping system implemented therein to supply breathable air from a source across the safety system is disclosed. The method includes executing a computing platform on a data processing device, and integrating the computing platform with a set of sensors associated with a number of components of the safety system. In accordance with the execution of the computing platform on the data processing device and the integration thereof with the set of sensors, the method also includes, through the data processing device, collecting a number of parameters of the number of components of the safety system and/or of access thereof through detection of the number of parameters via the set of sensors. The number of parameters includes one or more parameter(s) related to the breathable air and/or access of the breathable air. Further, the method includes monitoring the safety system and/or one or more component(s) of the number of components thereof based on the collected number of parameters.

In another aspect, a data processing device of a safety system of a structure having a fixed piping system implemented therein to supply breathable air from a source across the safety system is disclosed. The data processing device includes a memory including instructions associated with a computing platform stored therein, and a processor communicatively coupled to the memory. The processor executes the instructions associated with the computing platform to integrate the computing platform with a set of sensors associated with a number of components of the safety system, and, in accordance with the integration, collect a number of parameters of the number of components of the safety system and/or of access thereof through detection of the number of parameters via the set of sensors. The number of parameters includes one or more parameter(s) related to the breathable air and/or access of the breathable air. The processor also executes the instructions associated with the computing platform to monitor the safety system and/or one or more component(s) of the number of components thereof based on the collected number of parameters.

In yet another aspect, a safety system of a structure having a fixed piping system implemented therein to supply breathable air from a source across the safety system is disclosed. The safety system includes a data processing device executing a computing platform thereon, and a set of sensors associated with a number of components of the safety system. The execution of the computing platform on the data processing device integrates the computing platform with the set of sensors associated with the number of components of the safety system. In accordance with the execution of the computing platform on the data processing device and the integration thereof with the set of sensors, the data processing device collects a number of parameters of the number of components of the safety system and/or of access thereof through detection of the number of parameters via the set of sensors. The number of parameters includes one or more parameter(s) related to the breathable air and/or access of the breathable air. The data processing device also monitors the safety system and/or one or more component(s) of the number of components thereof based on the collected number of parameters.

Other features will be apparent from the accompanying drawings and from the detailed description that follows.

### BRIEF DESCRIPTION OF THE DRAWINGS

The embodiments of this invention are illustrated by way of example and not limitation in the figures of the accompanying drawings, in which like references indicate similar elements and in which:

FIG. 1 is a schematic and an illustrative view of a safety system associated with a structure, according to one or more embodiments.

FIG. 2 is a schematic view of the safety system of FIG. 1 with elements thereof integrated therewithin in detail, according to one or more embodiments.

FIG. 3 is a schematic view of the air monitoring system of the safety system of FIGS. 1-2, according to one or more embodiments.

FIG. 4 is a schematic view of an emergency air fill station of the safety system of FIGS. 1-2, according to one or more embodiments.

FIG. 5 is a schematic view of an air storage system of the safety system of FIGS. 1-2 according to one or more embodiments.

FIG. 6 is a schematic view of a computing platform relevant to the safety system of FIGS. 1-2 implemented through a server, according to one or more embodiments.

FIG. 7 is a schematic view of a data processing device of FIGS. 2-6, according to one or more embodiments.

FIG. 8 is an example user interface view of a component of the computing platform of FIG. 6 executing on the data processing device of FIGS. 2-7.

FIG. 9 is a process flow diagram detailing the operations involved in realizing a sensor integrated computing platform of a safety system of a structure for remote monitoring and access thereof, according to one or more embodiments.

Other features of the present embodiments will be apparent from the accompanying drawings and from the detailed description that follows.

### DETAILED DESCRIPTION

Example embodiments, as described below, may be used to provide a method, a device and/or a system of a sensor integrated computing platform of a safety system of a structure for remote monitoring and access thereof. Although the present embodiments have been described with reference to specific example embodiments, it will be evident that various modifications and changes may be made to these embodiments without departing from the broader spirit and scope of the various embodiments.

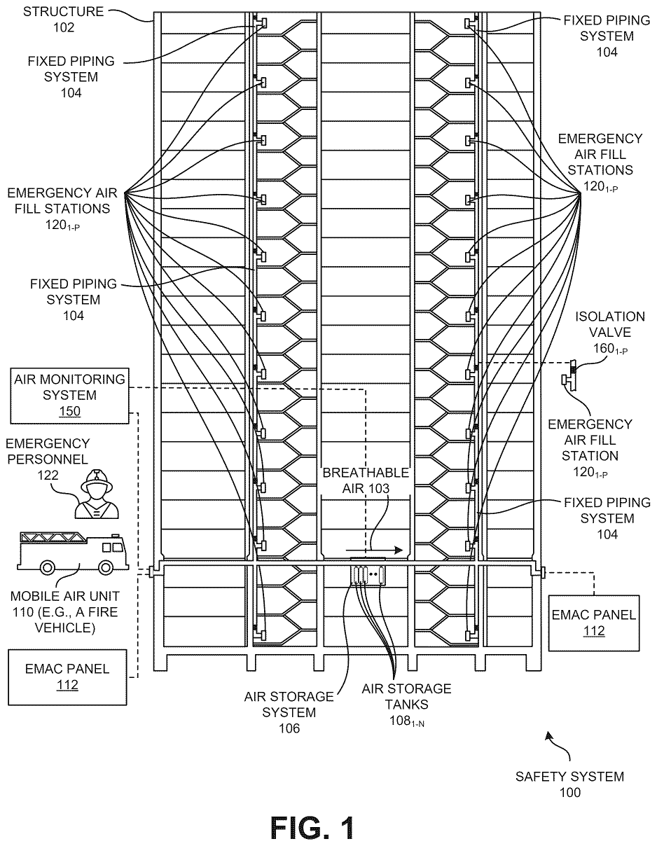

FIG. 1 shows a safety system 100 associated with a structure 102, according to one or more embodiments. In one or more embodiments, safety system 100 may be a Firefighter Air Replenishment System (FARS) to enable firefighters entering structure 102 in times of fire-related emergencies to gain access to breathable (e.g., human breathable) air (e.g., breathable air 103) in-house without the need of bringing in air bottles/cylinders to be transported up several flights of stairs of structure 102 or deep thereinto, or to refill depleted air bottles/cylinders that are brought into structure 102. In one or more embodiments, safety system 100 may supply breathable air provided from a supply of air tanks (to be discussed) stored in structure 102. When a fire department vehicle arrives at structure 102 during an emergency, breathable air supply typically may be provided through a source of air connected to said vehicle. In one or more embodiments, safety system 100 may enable firefighters to refill air bottles/cylinders thereof at emergency air fill stations (to be discussed) located throughout structure 102. Specifically, in some embodiments, firefighters may be able to fill air bottles/cylinders thereof at emergency air fill stations within structure 102 under full respiration in less than one to two minutes.

In one or more embodiments, structure 102 may encompass vertical building structures, horizontal building structures (e.g., shopping malls, hypermarts, extended shopping, storage and/or warehousing related structures), tunnels, marine craft (e.g., large marine vessels such as cruise ships, cargo ships, submarines and large naval craft, which may be “floating” versions of buildings and horizontal structures) and mines. Other structures are within the scope of the exemplary embodiments discussed herein. In one or more embodiments, safety system 100 may include a fixed piping system 104 permanently installed within structure 102 serving as a constant source of replenishment of breathable air 103. Fixed piping system 104 may be regarded as being analogous to a water piping system within structure 102 or another structure analogous thereto for the sake of imaginative convenience.

As shown in FIG. 1, fixed piping system 104 may distribute breathable air 103 across floors/levels of structure 102. For the aforementioned purpose, fixed piping system 104 may distribute breathable air 103 from an air storage system 106 (e.g., within structure 102) including a number of air storage tanks 108_{1-N} that serve as sources of pressurized/compressed air (e.g., breathable air 103). Additionally, in one or more embodiments, fixed piping system 104 may interconnect with a mobile air unit 110 (e.g., a fire vehicle) through an External Mobile Air Connection (EMAC) panel 112.

In one or more embodiments, EMAC panel 112 may be a boxed structure (e.g., exterior to structure 102) to enable the interconnection between mobile air unit 110 and safety system 100. For example, mobile air unit 110 may include an on-board air compressor to store and replenish pressurized/compressed air (e.g., breathable air analogous to breathable air 103) in air bottles/cylinders (e.g., utilizable with Self-Contained Breathing Apparatuses (SCBAs) carried by firefighters). Mobile air unit 110 may also include other pieces of air supply/distribution equipment (e.g., piping and/or air cylinders/bottles) that may be able to leverage the sources of breathable air 103 within safety system 100 through EMAC panel 112. Firefighters, for example, may be able to fill breathable air (e.g., breathable air 103, breathable air analogous to breathable air 103) into air bottles/cylinders (e.g., spare bottles, bottles requiring replenishment of breathable air) carried on mobile air unit 110 through safety system 100.

In FIG. 1, EMAC panel 112 is shown at two locations merely for the sake of illustrative convenience. In one or more embodiments, an air monitoring system 150 may be installed as part of safety system 100 to automatically track and monitor a parameter (e.g., pressure) and/or a quality (e.g., indicated by moisture levels, carbon monoxide levels) of breathable air 103 within safety system 100. FIG. 1 shows air monitoring system 150 as communicatively coupled to air storage system 106 and EMAC panel 112 merely for the sake of example. It should be noted that EMAC panel 112 may be at a remote location associated with (e.g., internal to, external to) structure 102. In one or more embodiments, for monitoring the parameters and/or the quality of breathable air within safety system 100, air monitoring system 150 include appropriate sensors and circuitries therein. For example, a pressure sensor (to be discussed) within air monitoring system 150 may automatically sense and record a pressure of breathable air 103 of safety system 100. Said pressure sensor may communicate with an alarm system that is triggered when the sensed pressure is outside a safety range. Also, in one or more embodiments, air monitoring system 150 may automatically trigger a shutdown of breathable air distribution through safety system 100 in case of impurity/contaminant (e.g., carbon monoxide) detection therethrough yielding levels above a safety/predetermined threshold.

In one or more embodiments, fixed piping system 104 may include pipes (e.g., constituted out of stainless steel tubing) that distribute breathable air 103 to a number of emergency air fill stations 120_{1-P} within structure 102. In one example implementation, each emergency air fill station 120_{1-P} may be located at a specific level of structure 102. If structure 102 is regarded as a vertical building structure, an emergency air fill station 120_{1-P} may be located at each of a basement level, a first floor level, a second floor level and so on. For example, emergency air fill station 120_{1-P} may be located at the end of the flight of stairs that emergency fighting personnel (e.g., firefighting personnel) need to climb to reach a specific floor level within the vertical building structure.

In one or more embodiments, an emergency air fill station 120_{1-P} may be a static location within a level of structure 102 that provides emergency personnel 122 (e.g., firefighters, emergency responders) with the ability to rapidly fill air bottles/cylinders (e.g., SCBA cylinders) with breathable air 103. In one or more embodiments, emergency air fill station 120_{1-P} may be an emergency air fill panel or a rupture containment air fill station. In one or more embodiments, proximate each emergency air fill station 120_{1-P}, safety system 100 may include an isolation valve 160_{1-P} to isolate a corresponding emergency air fill station 120_{1-P} from a rest of safety system 100. For example, said isolation may be achieved through the manual turning of isolation valve 160_{1-P} proximate the corresponding emergency air fill station 120_{1-P} or remotely (e.g., based on automatic turning) from air monitoring system 150. In one example implementation, air monitoring system 150 may maintain breathable air supply to a subset of emergency air fill stations 120_{1-P} via fixed piping system 104 through control of a corresponding subset of isolation valves 160_{1-P} and may isolate the other emergency air fill stations 120_{1-P} from the breathable air supply. It should be noted that configurations and components of safety system 100 may vary from the example safety system 100 of FIG. 1.

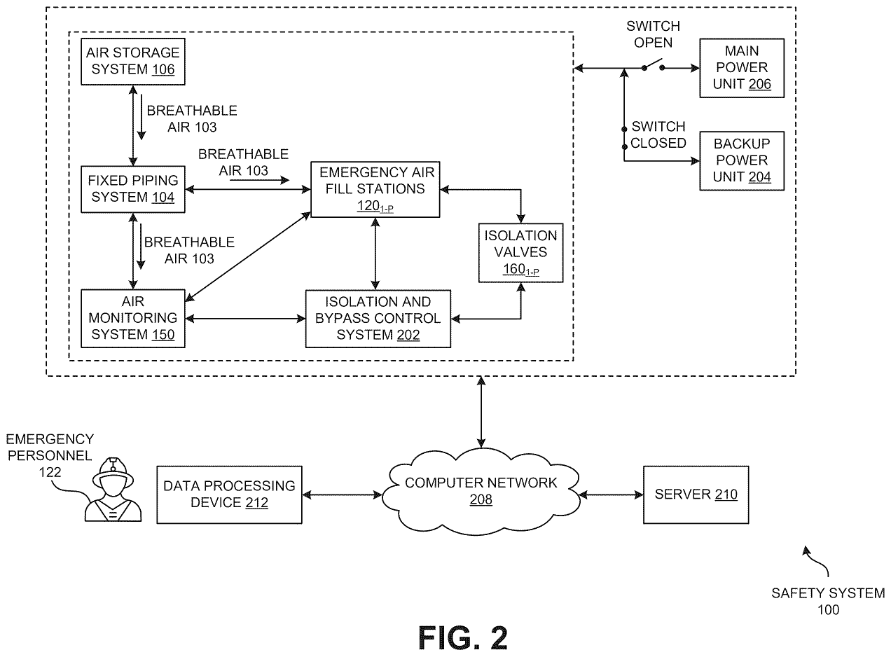

FIG. 2 shows safety system 100 with elements thereof integrated therewithin in detail, according to one or more embodiments. In one or more embodiments, safety system 100 may include air monitoring system 150 discussed above communicatively coupled to fixed piping system 104, to which emergency air fill stations 120_{1-P} are also coupled. In one or more embodiments, as seen above, the source of breathable air 103 may be air storage system 106. In one or more embodiments, safety system 100 may also include an isolation and bypass control system 202 that is constituted by a set of electrical, mechanical and/or electronic components working together to automatically include and/or bypass one or more emergency air fill station(s) 120_{1-P} based on detection of anomalous air parameters, as will be discussed below. For the aforementioned purpose, in one or more embodiments, isolation valve(s) 160_{1-P} associated with the aforementioned emergency air fill stations 120_{1-P} may be controlled (e.g., by opening or closing one or more of said isolation valves 160_{1-P}) by isolation and bypass control system 202. In other words, in one or more embodiments, the one or more isolation valves 160_{1-P} may be opened or closed to control the flow of breathable air 103 from the source thereof to a corresponding one or more emergency air fill stations 120_{1-P} in response to detection of anomalous air parameters or collection of the air parameters (e.g., parameters 304, parameters 404, parameters 508 to be discussed below).

Further, in one or more embodiments, safety system 100 may include a backup power unit 204 (e.g., an electrical power system with electronic integration) to ensure uninterrupted power to components of safety system 100 during emergencies (e.g., a power cut, a mains power issue, a fire accident effected power issue). For the aforementioned purpose, in one or more embodiments, backup power unit 204 may be switched on in the case of a power related emergency with respect to a main power unit 206 (e.g., Alternating Current (AC) mains power, Direct Current (DC) power) associated with safety system 100.

In one or more embodiments, one or more or all of the abovementioned components of safety system 100 may be integrated with sensor(s) to detect parameters of use therewithin. In one or more embodiments, one or more of the aforementioned components may be communicatively coupled through a computer network 208 (e.g., a Local Area Network (LAN), a Wide Area Network (WAN), a cloud computing network, the Internet, a short-range communication network based on Bluetooth®, WiFi® and the like) to a remote server 210 (e.g., a network of servers, a single server, a distributed network of servers, a command room server associated with safety system 100 and so on). As will be discussed below, in one or more embodiments, server 210 may obtain said parameters of use and other data from safety system 100 and perform analysis (e.g., predictive, non-predictive) thereof.

In addition, in one or more embodiments, safety system 100 may include a data processing device 212 (e.g., a mobile phone, a tablet, an iPad®, a laptop, a desktop) also communicatively coupled to one or more components or each component of safety system 100 and server 210 through computer network 208. Thus, in one or more embodiments, one or more components or each component of safety system 100 may have interfaces (not explicitly shown) for wireless communication through computer network 208. Also, as will be discussed below, in one or more embodiments, wherever possible, elements (e.g., handheld Thermal Imaging Cameras (TICs), portable TICs, aerial TICs, video cameras, output audio devices, output light devices, one or more or all sensors discussed herein) may be Internet of Things (IoT) devices capable of collecting and feeding data to server 210 through computer network 208. In one or more embodiments, IoT devices (or IoT enabled devices) may be devices and/or components with programmable hardware that can transmit data over computer networks (e.g., computer network 208 such as the Internet and/or other networks); said IoT devices may include or be associated with edge devices (not shown) to control data flow at the boundaries to computer network 208.

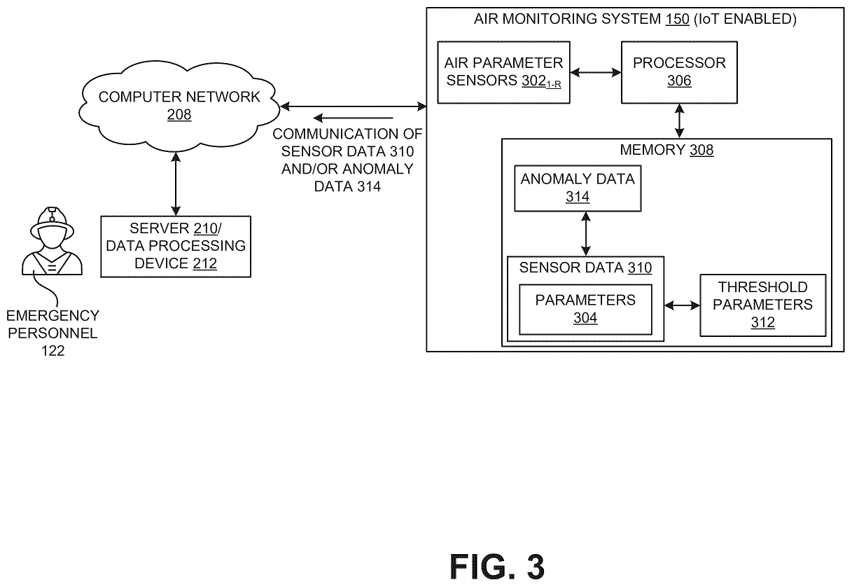

FIG. 3 shows air monitoring system 150, according to one or more embodiments. In one or more embodiments, air monitoring system 150 may include one or more air parameter sensors 302_{1-R} configured to sense parameters 304 associated with breathable air 103 such as pressure, temperature, oxygen content, carbon monoxide content, hydrocarbon content and moisture content; other parameters (e.g., air quality parameter(s), non-air quality parameter(s)) are within the scope of the exemplary embodiments discussed herein. In one or more embodiments, air monitoring system 150 may include a processor 306 (e.g., a microcontroller, a processor core, a single processor) communicatively coupled to a memory 308 (e.g., a volatile and/or a non-volatile memory); FIG. 3 shows air parameter sensors 302_{1-R} interfaced with processor 306. In one or more embodiments, data sensed by the aforementioned air parameter sensors 302_{1-R} may be part of sensor data 310 stored in memory 308; parameters 304 sensed may be part of sensor data 310.

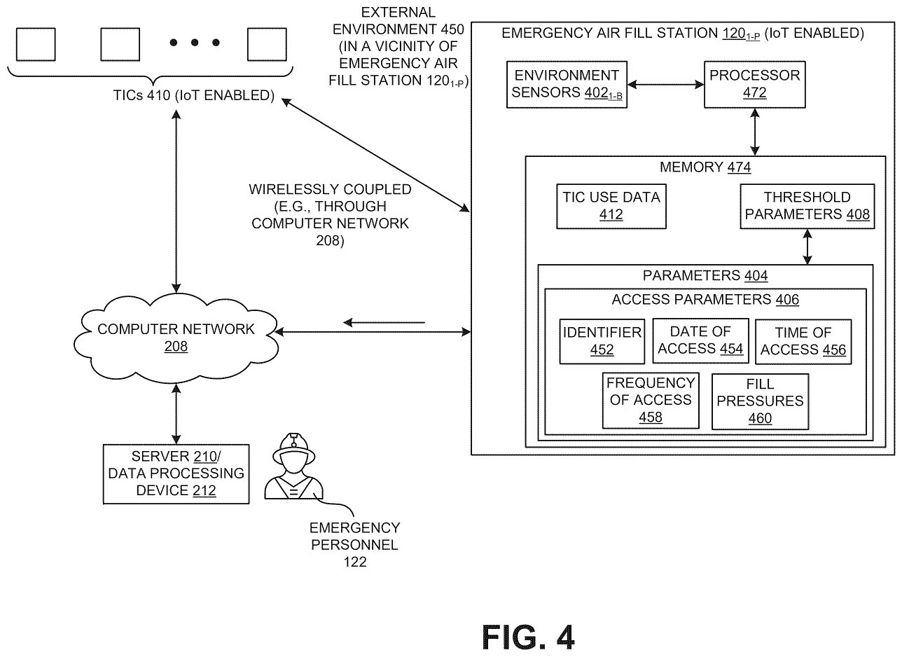

In one or more embodiments, threshold values/ranges (e.g., threshold parameters 312) for parameters 304 sensed may also be stored in memory 308. In one or more embodiments, detecting through processor 306 in conjunction with one or more air parameter sensors 302_{1-R} that one or more parameters 304 is outside (e.g., below, above, outside) threshold parameters 312 may cause communication of anomalies (e.g., detected anomaly data 314 stored in memory 308) to server 210 through computer network 208 in accordance with the IoT capabilities discussed above. FIG. 4 shows an emergency air fill station 120_{1-P}, according to one or more embodiments. Again, in one or more embodiments, emergency air fill station 120_{1-P} may include one or more environment sensors 402_{1-B} integrated therewith configured to sense parameters 404 (e.g., temperature, ambient light) of an environment (e.g., external environment 450) in an immediate vicinity of emergency air fill station 120_{1-P}. In one or more embodiments, environment sensors 402_{1-B} may also sense access (e.g., access parameters 406 that are part of parameters 404 in FIG. 4) of emergency air fill station 120_{1-P} by emergency personnel 122 (e.g., maintenance personnel, firefighters, emergency responders). Example access parameters 406 may include but are not limited to identifier 452 of emergency personnel 122, date of access 454 mapped to identifier 452, time of access 456 mapped to identifier 452, a frequency of access 458 and fill pressures 460 (e.g., pressures to which breathable air 103 is filled in air bottles/cylinders discussed above) mapped to identifier 452, time of access 456 and/or date of access 454.

In one or more embodiments, again based on sensed parameters 404 being outside (e.g., more than, less than, outside a range) threshold values/ranges (e.g., threshold parameters 408) based on the IoT capabilities discussed herein, anomalies in parameters 404 may be detected and collected at emergency air fill station 120_{1-P} and transmitted to server 210 through computer network 208. In one or more embodiments, as shown in FIG. 4, emergency air fill station 120_{1-P} may include a processor 472 (e.g., a microcontroller, a processor core, a single processor) communicatively coupled to a memory 474 (e.g., a volatile and/or a non-volatile memory). In one or more embodiments, environment sensors 402_{1-B} may be interfaced with processor 472 and all of the abovementioned data/parameters may be stored in memory 474, as shown in FIG. 4.

FIG. 4 also shows TICs 410 as part of safety system 100 and in external environment 450 of emergency air fill station 120_{1-P}, according to one or more embodiments. In one or more embodiments, TICs 410 may be infrared cameras that sense infrared energy of objects to render images/video frames thereof corresponding to surface temperatures of said objects. In one or more embodiments, emergency personnel 122 may employ said TICs 410 to detect obstacles on the paths to/around emergency air fill stations 120_{1-P} under low visibility; this may enable emergency personnel 122 perform rescue operations efficiently. As discussed and implied above, TICs 410 may be integrated with IoT capabilities to transmit data to server 210 through computer network 208. Said data may be part of access parameters 406 or separate data (e.g., TIC use data 412) transmitted to server 210.

It should be noted that the sensing, detection and/or transmission of data to server 210 discussed above with regard to emergency air fill station 120_{1-P} may also be performed at a device external to emergency air fill station 120_{1-P}. In such implementations, the external device itself may obviously be a component of safety system 100 with IoT/wireless communication capabilities. All reasonable variations are within the scope of the exemplary embodiments discussed herein.

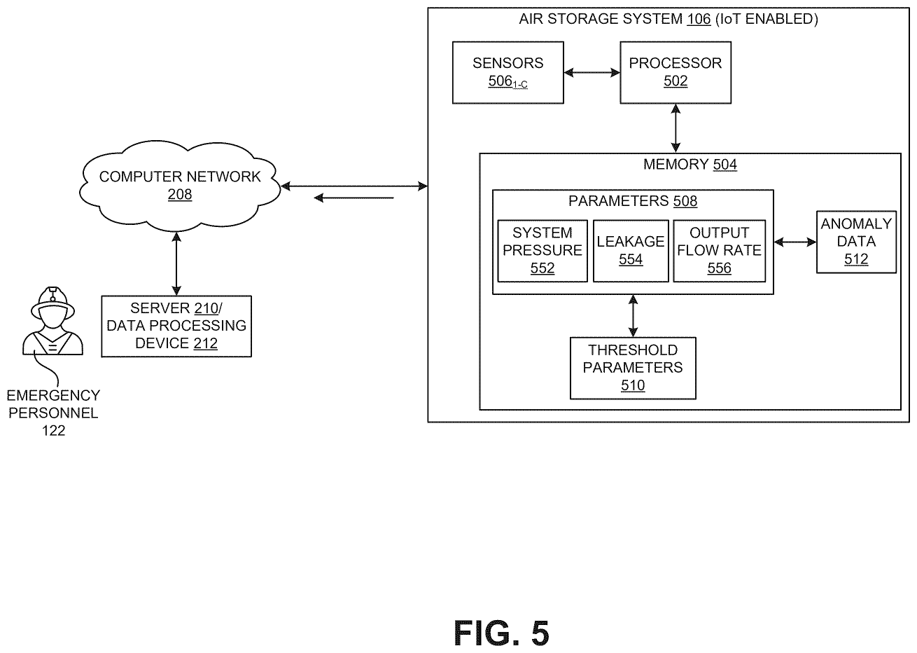

FIG. 5 shows air storage system 106, according to one or more embodiments. Again, as discussed above, in one or more embodiments, air storage system 106 may have IoT/wireless communication capabilities embedded therein or in a device external thereto that is communicatively coupled to air storage system 106. In one or more embodiments, air storage system 106 may include a processor 502 (e.g., a microcontroller, a processor core, a single processor) communicatively coupled to a memory 504 (e.g., a volatile and/or a non-volatile memory). Again, in one or more embodiments, air storage system 106 may include one or more sensors 506_{1-C} configured to sense parameters (e.g., parameters 508 stored in memory 504) associated with air storage system 106; sensors 506_{1-C} are shown interfaced with processor 502 Example parameters 508 sensed may include but are not limited to system pressure 552 (e.g., pressure at which breathable air 103 is output from air storage system 106), leakage 554 (e.g., leakage of breathable air 103 from air storage tanks 108_{1-N}) and output flow rate 556 (e.g., rate of flow of breathable air 103 out of air storage system 106). In one or more embodiments, parameters 508 may be transmitted to server 210 through computer network 208 for processing and/or analysis thereat.

Again, in one or more embodiments, anomalies based on parameters 508 being outside thresholds/ranges (e.g., threshold parameters 510 stored in memory 504) may be detected through sensors 506_{1-C} (e.g., flow rate sensors, pressure sensors). FIG. 5 shows anomaly data 512 relevant to the aforementioned detected anomalies also transmitted to server 210 through computer network 208, according to one or more embodiments.

It should be noted that FIGS. 3-5 merely relate to example components of safety system 100 with which sensors/IoT devices are integrated and that integration of sensors/IoT devices with any other component (e.g., backup power unit 204 to sense frequency and/or duration of use thereof, isolation and bypass control system 202 to sense a frequency of bypass/isolation of emergency air fill stations 120₁₋ₚ, turning on/off of isolation valves 160_{1-P} and so on) thereof conceivable is within the scope of the exemplary embodiments discussed herein. Referring back to FIG. 4, identifier 452 within access parameters 406 relevant to access of emergency air fill station 120_{1-P} may also encompass a key fob based identification, a Radio Frequency Identification (RFID) based access, a Non-Fungible Token (NFT) based access, keys and/or access through an application component (e.g., component 706 to be discussed below) executing on data processing device 212. All reasonable variations are within the scope of the exemplary embodiments discussed herein.

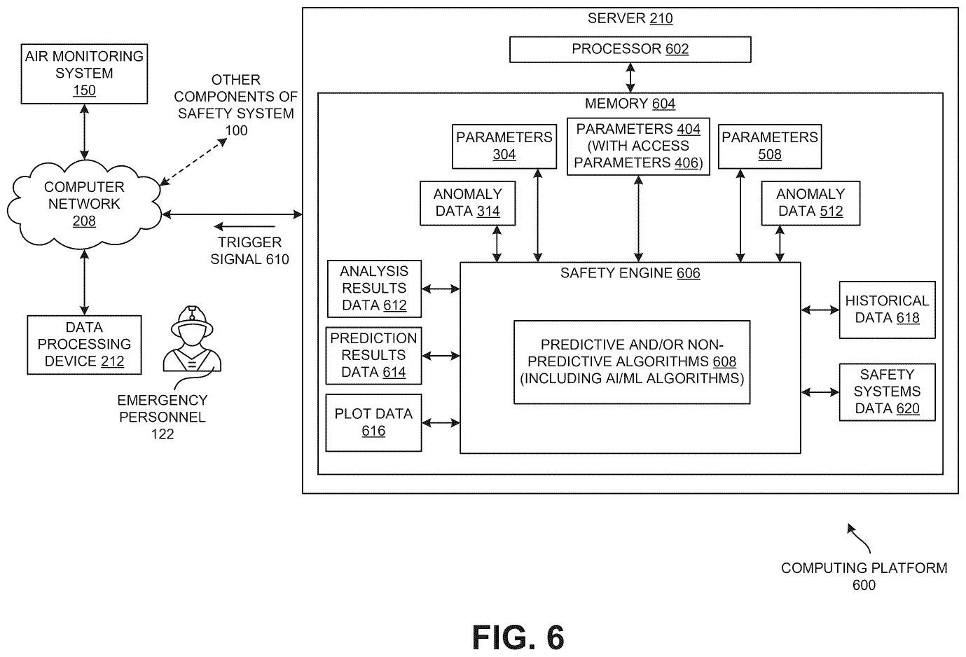

FIG. 6 shows a computing platform 600 relevant to the FARS of safety system 100 implemented through server 210, according to one or more embodiments. In one or more embodiments, server 210 may be a distributed (e.g., across a cloud) network of servers, a cluster of servers or a standalone server. As shown in FIG. 6, server 210 may include a processor 602 (e.g., a processor core, a network of processors, a single processor), communicatively coupled to a memory 604 (e.g., a volatile and/or a non-volatile memory). In one or more embodiments, memory 604 may include a safety engine 606 associated with the FARS stored therein and executable through processor 602. FIG. 6 shows memory 604 as including data (e.g., detected, sensed, anomalies) from one or more components of safety system 100; the limited amount of data shown must not be considered as limiting the scope of the exemplary embodiments discussed herein. In one or more embodiments, safety engine 606 may have one or more predictive and/or non-predictive algorithms (e.g., predictive and/or non-predictive algorithms 608) including Artificial Intelligence (AI)/Machine Learning (ML) based algorithms stored therein.

In one or more embodiments, execution of predictive and/or non-predictive algorithms 608 through processor 602 may involve taking the abovementioned data and profiling the FARS implemented as safety system 100. It should be noted that each of the aforementioned data (e.g., parameters 304, parameters 404, access parameters 406, parameters 508, anomaly data 314, anomaly data 512) may be real-time data from elements of safety system 100. In one or more embodiments, analysis of the data may result in beneficial decision making with regard to maintenance of safety system 100, safety of safety system 100 and/or efficiency thereof. For example, anomalies discussed above may be analyzed based on date, time and/or frequency thereof to predict that a specific duration of time in a winter season is associated with diminished characteristics of a component of safety system 100. All possible analyses are within the scope of the exemplary embodiments discussed herein.

In one or more embodiments, server 210 may also be utilized to remotely test and/or trigger operations of one or more components of safety system 100. FIG. 6 shows a trigger signal 610 communicated to air monitoring system 150 to get data thereof discussed above from processor 306, according to one or more embodiments. In some implementations, the components of safety system 100 may automatically transmit data thereof to server 210 and in some others, server 210 may transmit trigger signals (e.g., trigger signal 610) therefor. FIG. 6 also shows results of analysis/prediction through safety engine 606 as analysis results data 612, prediction results data 614 and plot data 616 (e.g., related to graphically plotting the results of analyses). Further, FIG. 6 shows data processing device 212 communicatively coupled to server 210 through computer network 208 as part of computing platform 600, according to one or more embodiments.

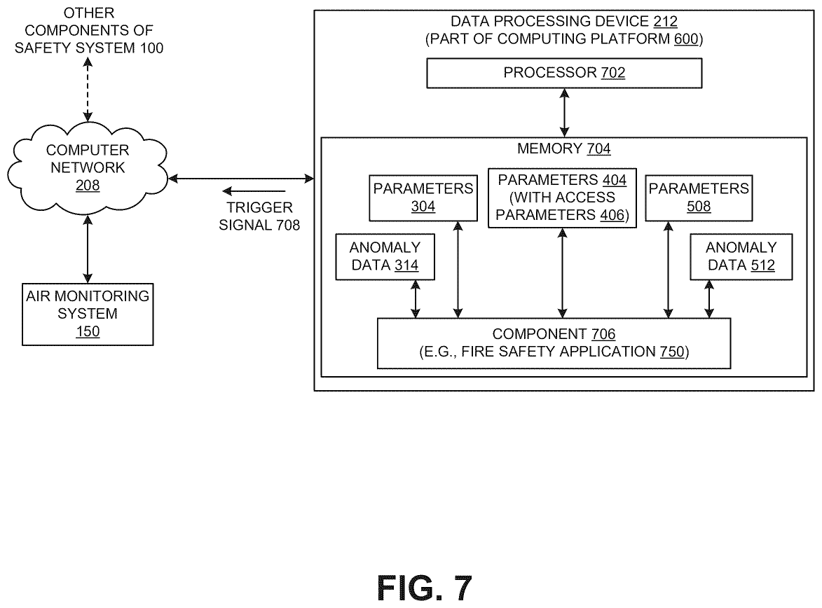

FIG. 7 shows data processing device 212 (e.g., a mobile phone, a tablet, a smart device, a laptop) in detail, according to one or more embodiments. In one or more embodiments, again, data processing device 212 may include a processor 702 (e.g., a single processor, a processor core) communicatively coupled to a memory 704 (e.g., a volatile and/or a non-volatile memory). In one or more embodiments, memory 704 may include a component 706 of safety engine 606 stored therein and enabled/provided through processor 602 of server 210. FIG. 7 shows component 706 as a fire safety application 750 merely for example purposes. Again, in one or more embodiments, access to the data of one or more components of safety system 100 may be available to data processing device 212 via component 706 (e.g., through computer network 208 via safety engine 606 of server 210). FIG. 7 also shows capabilities to control components of safety system 100 through data processing device 212 via trigger signals; FIG. 7 specifically shows a trigger signal 708 to initiate collection of data from air monitoring system 150 merely for example purposes. Again, in some implementations, data may be automatically communicated to data processing device 212 and in some others, data processing device 212 may trigger (e.g., through trigger signal 708) collection thereof.

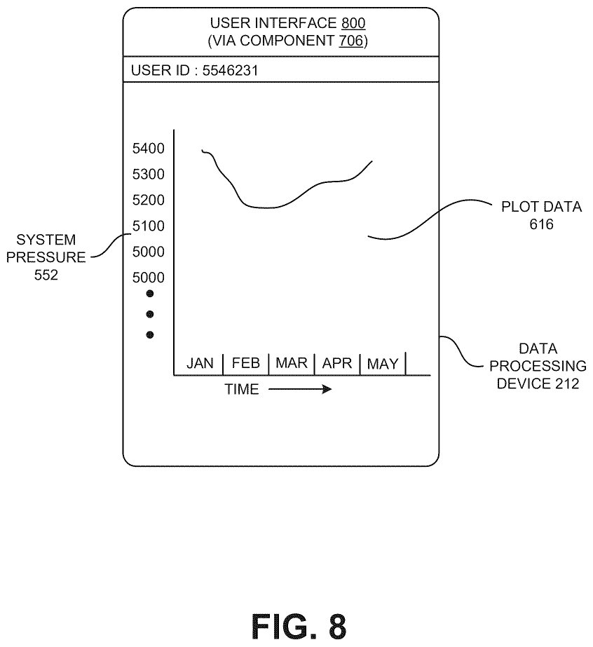

In one or more embodiments, access of emergency air fill station 120_{1-P} through component 706 may cause collection of identifier 452 discussed above as part of access parameters 406. FIG. 8 shows an example user interface 800 provided via component 706 (e.g., fire safety application 750) executing on data processing device 212. Here, user interface 800 shows plot data 616 discussed above that may be based on one or more examples of data discussed above and/or analysis results data 612; plot data 616 in FIG. 8 is an evolution of system pressure 552 over a scale of time. Obviously, emergency personnel 122 and/or other users associated with data processing device 212 may access user interface 800 after authentication thereof via computing platform 600 and/or navigating to user interface 800. All reasonable variations are within the scope of the exemplary embodiments discussed herein.

Thus, exemplary embodiments discussed herein provide for an integrated FARS computing platform (e.g., computing platform 600) that enables collection and/or analysis of real-time data from one or more components of safety system 100 and/or control (e.g., remotely; in one scenario, one or more isolation valves 160_{1-P} may be opened or closed through server 210/data processing device 212/isolation and bypass control system 202 to control the flow of breathable air 103 from the source thereof to a corresponding one or more emergency air fill stations 120_{1-P} in r esponse to collection of air parameters (e.g., parameters 304, parameters 404, parameters 508)) thereof. Further, the integrated FARS computing platform may provide for profiling of safety system 100 and/or emergency personnel 122 and/or remote management of requirements associated with safety system 100. For example, the profiling may involve utilizing (e.g., through safety engine 606) historical data (e.g., historical data 618 stored in memory 604 of server 210) from one or more components of safety system 100 and/or generic safety systems data (e.g., safety systems data 620 stored in memory 604 of server 210) from one or more safety systems other than safety system 100 to arrive at parts of analysis results data 612, prediction results data 614 and/or plot data 616. Again, as discussed above, in one or more embodiments, the integrated FARS computing platform may provide for quick decision making on the part of maintenance personnel, administrative personnel and/or emergency personnel (e.g., emergency personnel 122) associated with safety system 100; statistical analyses and/or data gathering and/or predictive and/or non-predictive analyses may also be enabled through the integrated FARS computing platform.

Also, in one or more embodiments, analogous analyses and/or prediction may also be performed at data processing device 212 based on enablement thereof through component 706. Further, it should be noted that detection of anomalies (e.g., anomaly data 314, anomaly data 512) may be performed through server 210 based on execution of safety engine 606 discussed above instead of or in addition to the detection thereof at the respective components. Last but not the least, as computing platform 600 may be enabled through the execution of safety engine 606, which, in turn, may enable component 706, both safety engine 606 and component 706 may be interpreted as computing platform 600 executing on server 210 and data processing device 212 respectively. All reasonable variations are within the scope of the exemplary embodiments discussed herein.

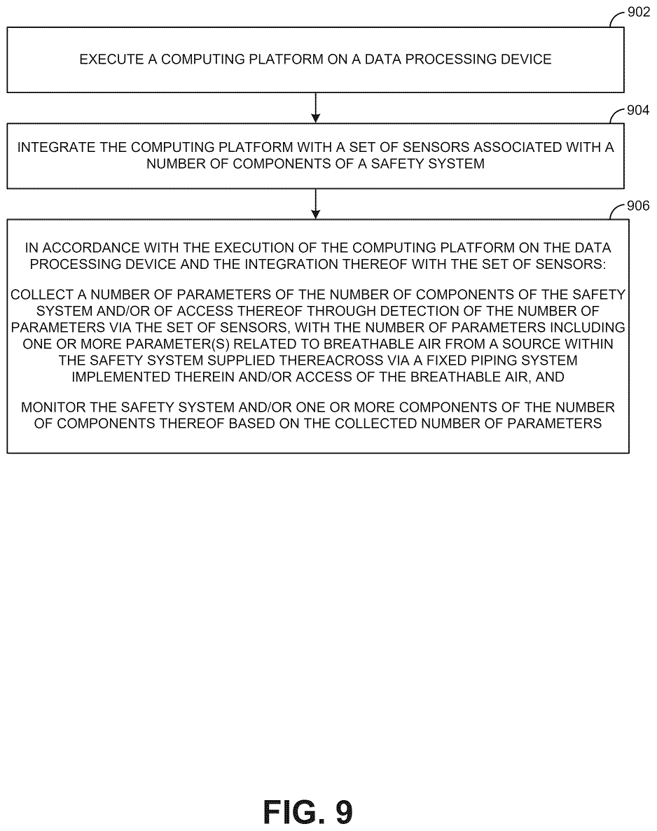

FIG. 9 shows a process flow diagram detailing the operations involved in realizing a sensor integrated computing platform (e.g., computing platform 600, safety engine 606, component 706) of a safety system (e.g., safety system 100) of a structure (e.g., structure 102) for remote control and access thereof, according to one or more embodiments. In one or more embodiments, operation 902 may involve executing the computing platform on a data processing device (e.g., server 210, data processing device 212). In one or more embodiments, operation 904 may involve integrating the computing platform with a set of sensors (e.g., air parameter sensors 302_{1-R}, environment sensors 402_{1-B}, sensors 506_{1-C}) associated with a number of components (e.g., air monitoring system 150, emergency air fill station 120_{1-P}, air storage system 106) of the safety system.

In one or more embodiments, operation 906 may then involve, in accordance with the execution of the computing platform on the data processing device and the integration thereof with the set of sensors, through the data processing device, collecting a number of parameters (e.g., parameters 304, parameters 404, parameters 508) of the number of components of the safety system and/or of access (e.g., access parameters 406) thereof through detection of the number of parameters via the set of sensors. In one or more embodiments, the number of parameters may include one or more parameter(s) (e.g., system pressure 552, leakage 554, output flow rate 556, fill pressures 460) related to breathable air (e.g., breathable air 103) from a source (e.g., air storage system 106) within the safety system supplied thereacross via a fixed piping system (e.g., fixed piping system 104) implemented therein and/or access of the breathable air.

In one or more embodiments, operation 906 may also involve monitoring the safety system and/or one or more component(s) of the number of components thereof based on the collected number of parameters.

Although the present embodiments have been described with reference to specific example embodiments, it will be evident that various modifications and changes may be made to these embodiments without departing from the broader spirit and scope of the various embodiments.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

1-20. (canceled)

21. A firefighter air replenishment system (FARS), comprising:
a safety system, comprising:
a source of air comprising tanks storing breathable air at at least 4500 pounds per square inch (PSI); and
a fixed piping system coupled with the source of air;
a plurality of sensors coupled with the safety system; and
a data processing device coupled with the plurality of sensors, the data processing device comprising one or more processors to:
detect one or more parameters of the breathable air; and
manage access to the breathable air according to the one or more parameters.

22. The FARS of claim 21, comprising:
the plurality of sensors comprise one or more air parameter sensors to sense at least one of: pressure, temperature, oxygen content, carbon monoxide content, hydrocarbon content, and moisture content of the breathable air.

23. The FARS of claim 21, comprising:
the one or more processors are to detect at least one anomaly of the one or more parameters based at least on a comparison of the one or more parameters with a corresponding threshold.

24. The FARS of claim 21, comprising:
an air monitoring system coupled with the fixed piping system, the air monitoring system comprising at least one sensor of the plurality of sensors.

25. The FARS of claim 24, comprising:
an air monitoring system coupled with the fixed piping system, the air monitoring system comprising at least one sensor of the plurality of sensors, the air monitoring system to trigger a shutdown of breathable air distribution in response to detection of an impurity or contaminant at a level above a predetermined threshold.

26. The FARS of claim 21, comprising:
a plurality of emergency air fill stations coupled with the fixed piping system and distributed throughout a structure, each emergency air fill station of the plurality of emergency air fill stations to provide access to the breathable air for filling air cylinders.

27. The FARS of claim 21, comprising:
a plurality of emergency air fill stations; and
a plurality of isolation valves, each isolation valve of the plurality of isolation valves being coupled with a corresponding emergency air fill station of the plurality of emergency air fill stations.

28. The FARS of claim 21, comprising:
the one or more processors are to control one or more isolation valves of a plurality of isolation valves to selectively include or bypass one or more of emergency air fill stations of a plurality of emergency air fill stations based on detection of one or more anomalous parameters.

29. The FARS of claim 21, comprising:
the plurality of sensors comprise one or more environment sensors integrated with at least one emergency air fill station of a plurality of emergency air fill stations.

30. The FARS of claim 21, comprising:
the plurality of sensors are to detect one or more access parameters comprising at least one of: an identifier of emergency personnel accessing the breathable air, a date of access, a time of access, a frequency of access, and fill pressures.

31. The FARS of claim 21, comprising:
the plurality of sensors are to sense the one or more parameters to include least one of system pressure, leakage, and output flow rate of the breathable air.

32. A method, comprising:
detecting, by one or more processors of a data processing device coupled with a plurality of sensors, one or more parameters of breathable air stored by a plurality of tanks; and
managing, by the one or more processors, access to the breathable air according to the one or more parameters.

33. The method of claim 32, comprising:
obtaining, by the one or more processors from the plurality of sensors, the one or more air parameters as at least one of: pressure, temperature, oxygen content, carbon monoxide content, hydrocarbon content, and moisture content of the breathable air.

34. The method of claim 32, comprising:
detecting, by the one or more processors, at least one anomaly of the one or more parameters based at least on a comparison of the one or more parameters with a corresponding threshold.

35. The method of claim 32, comprising:
the breathable air is distributed via a fixed piping system to a plurality of emergency air fill stations distributed throughout a structure, each emergency air fill station of the plurality of emergency air fill stations to provide access to the breathable air for filling air cylinders.

36. The method of claim 32, comprising:
controlling, by the one or more processors, one or more isolation valves of a plurality of isolation valves to selectively include or bypass one or more emergency air fill stations of a plurality of emergency air fill stations based on detection of one or more anomalous parameters.

37. The method of claim 32, comprising:
the plurality of sensors comprise one or more environment sensors integrated with at least one emergency air fill station of a plurality of emergency air fill stations.

38. The method of claim 32, comprising:
detecting, by the plurality of sensors, one or more access parameters comprising at least one of: an identifier of emergency personnel accessing the breathable air, a date of access, a time of access, a frequency of access, and fill pressures.

39. The method of claim 32, comprising:
the one or more parameters include at least one of: system pressure, leakage, and output flow rate of the breathable air.

40. The method of claim 32, comprising:
triggering, by the one or more processors, a shutdown of breathable air distribution in response to detection of an impurity or contaminant at a level above a predetermined threshold.
